(19) United States
(12) Patent Application Publication
(10) Pub. No.: US 2004/0197402 A1
(43) Pub. Date: Oct. 7, 2004

(54) Oxcarbazepine dosage forms

(76) Inventors: Ashish Sehgal, Delhi (IN); Anupam Trehan, New Delhi, Delhi (IN); Vinod Kumar Arora, New Delhi, Delhi (IN)

Correspondence Address: RANBAXY INC., 600 COLLEGE ROAD EAST, SUITE 2100, PRINCETON, NJ, 08540, US

(21) Appl. No.: 10/478,046

(22) PCT Filed: May 20, 2002
(86) PCT No.: PCT/IB02/01720

(30) Foreign Application Priority Data
May 18, 2001 (IN) 596/DEL/2001

(51) Int. Cl. A61K 31/55; A61K 9/20

(52) U.S. Cl. 424/465; 514/217

(57) ABSTRACT

The present invention relates to dosage forms of oxcarbazepine for oral administration and to the process for the preparation of such dosage forms.

### FIELD OF THE INVENTION

The present invention relates to dosage forms of oxcarbazepine for oral administration and to the process for the preparation of such dosage forms.

### BACKGROUND OF THE INVENTION

Drug insolubility is one of the major challenges in the development of many pharmaceutical products. Over one third drugs of the listed in the US Pharmacopoeia and about fifty percent of New Chemical Entities are insoluble or poorly soluble in water. The result, is that many drugs are marketed as sub-optimal formulations, after giving poor or erratic bioavailability or a greater risk of adverse side effects. Oxcarbazepine, 10, 11-dihydro-10-oxo-5H-dibenz [b,f], azepine-5-carboxamide, a widely used antiepileptic drug has poor solubility in water.

One of the earlier attempts to enhance the dissolution rate and availability/bioavailability of oxcarbazepine relied on particle size reduction of the pure oxcarbazepine to an order of 2 to 12 μm. While size reduction to 2-12 μm particle size does enhance absorption over the use of unmicronized or bigger particle size material, it still requires special equipment/machines like air-jet mill or impact mill, ball mill, vibration mill, mortar mill or pin mill. All these are high energy consuming machines, therefore, size reduction to 2-12 μm range is not only time consuming but also a costly process. Further, the micronized particles tend to agglomerate, thus diminishing both, the solubility and bioavailability of the drug. A PCT application, WO 98/35681 is an illustration of oxcarbazepine composition for oral administration employing micronized drug particles of 2 to 12 μm ranges.

Oxcarbazepine tablets are also known to undergo a color change during storage. The discoloration is caused by the formation of a minor amount of an oxidation product “diketoiminodibenzyl: 10, 11-dihydro-5H-dibenzo [b,f] azepine-10,11-dione. This oxidation product is considered to be pharmacologically harmless. However, the color change is not generally pharmaceutically desirable.

U.S. Pat. Nos. 5,472,714 and 5,695,782 describe color stable oxcarbazepine tablets. The colour stability has been achieved by providing double coating to the tablets. Oxcarbazepine tablets described therein are provided with hydrophilic, permeable inner layer containing white pigments and further a hydrophilic, permeable outer layer containing white pigments in combination with iron (II) oxide pigments. U.S. Pat. Nos. 5,472,714; 5,695,782 and the PCT application WO 98/35681 show the use of iron oxide pigment. The U.S.—FDA permits oral ingestion of only 5 mg iron daily. Furthermore, the coatings add to the cost and time and to the complexity in manufacturing.

### SUMMARY OF THE INVENTION

It is an object of the present invention to provide improved formulations and a simple and cost effective process for the preparation of pharmaceutically elegant oxcarbazepine dosage forms.

Accordingly, the present invention provides a dosage form for oral administration comprising oxcarbazepine and a wetting agent.

According to another aspect of the present invention, it relates to a process for the preparation of oxcarbazepine dosage forms, for oral administration comprising the steps of:

a) treating oxcarbazepine alone or a blend of oxcarbazepine and other pharmaceutical excipients with a wetting agent; and

b) formulating into suitable dosage form.

The present invention provides a simple, less time consuming and economical process of preparing oxcarbazepine tablets. As the target dissolution (similar to the marketed form) profile in the present invention is obtained by the use of a wetting agent rather than the particle size reduction. Use of a wetting agent reduces the surface tension of water and therefore increases adhesion of water to the oxcarbazepine surface. Improved wettability is observed as a lower contact angle between the oxcarbazepine and water which in turn results in improved dissolution. The use of a wetting agent may also be useful in improving the bioavailability of oxcarbazepine.

To ensure an external homogeneity of the product, the coloring agent is added during the compression. This provides the advantage of making the process further simple and cost effective as no coating is required. Furthermore, the coloring agent used is other than iron oxide (which has a limited daily intake).

The term “treating” means mixing/granulating either oxcarbazepine alone or a blend of oxcarbazepine and other pharmaceutical excipients with a sufficient amount of a wetting agent.

The wetting treatment is accomplished either

a) by forming a slurry, wet granulation or a paste mixture of the oxcarbazepine or a blend of oxcarbazepine and other pharmaceutical excipients with the wetting solution containing wetting agent; or

b) by mixing the wetting agent with oxcarbazepine or a blend of oxcarbazepine and other pharmaceutical agents and then granulating.

The wetting treatment can be achieved either with small incremental additions of the wetting solution or a large single shot treatment. The purpose of the wetting treatment is to distribute wetting agent uniformly to the surfaces of the drug particles of oxcarbazepine. This could also be achieved by dry blending oxcarbazepine and wetting agents, and then compacting or slugging the blend.

The “wetting agent” of the present invention may be selected from anionic, cationic or non-ionic surface active agents or surfactants. Suitable anionic surfactants include those containing carboxylate, sulfonate, and sulfate ions such as sodium lauryl sulfate (SLS), sodium laurate, dialkyl sodium sulfosuccinates particularly bis-(2-ethylhexyl) sodium sulfosuccinate, sodium stearate, potassium stearate, sodium oleate and the like. Suitable cationic surfactants include those containing long chain cations, such as benzalkonium chloride, bis-2-hydroxyethyl oleyl amine or the like. Suitable non-ionic surfactants include polyoxyethylene sorbitan fatty acid esters, fatty alcohols such as lauryl, cetyl and stearyl alcohols; glyceryl esters such as the naturally occurring mono-, di-, and tri-glycerides; fatty acid esters of fatty alcohols and other alcohols such as propylene glycol, polyethylene glycol, sorbitan, sucrose, and cholesterol.

The wetting agent should generally be used in an amount which is sufficient to wet. This amount would vary with the type of surface active agent used and also the method by which it is added. Normally, small increment treatments would require lower amounts of wetting agent than large or single shot treatments.

The wetting agent when used with oxcarbazepine having a median particle size of about 20 μm to about 50 μm with a maximum residue of about 10% on a 45 μm to up to 100 μm sieve (which could easily be achieved by milling using conventional equipment such as Cad Mill or Multi Mill) gives the best results.

The other excipients of this invention may be selected from amongst the diluents, binders, disintegrants, lubricants, glidants, colouring agents, flavouring agents and sweeteners, which are chemically and physically compatible with oxcarbazepine.

Diluents of this invention may be selected from any such pharmaceutically acceptable excipients, which give bulk to the oxcarbazepine composition; preferably those diluents may be selected from starch, microcrystalline cellulose, lactose, glucose, mannitol, alginates, alkali earth metal salts, clays or polyethylene glycols.

Binders of this invention may be selected from any such pharmaceutically acceptable excipient, which have cohesive properties to act as binders. Preferably, those excipients are starch, microcrystalline cellulose, highly dispersed silica, mannitol, lactose, polyethylene glycol, cross-linked polyvinylpyrrolidone, cross-linked carboxymethyl cellulose, hydroxypropyl methyl cellulose and hydroxy propyl cellulose.

Disintegrants preferred for the present invention may be selected from starches or modified starches such as sodium starch glycolate, corn starch, potato starch or pregelatinized starch, clays such as bentonite, montmorillonite or veegum; celluloses such as microcrystalline cellulose, hydroxypropyl cellulose or carboxymethyl cellulose, algins such as sodium alginate or alginic acid; cross-linked cellulose such as croscarmellose sodium, gums such as guar gum or xanthan gum; cross-linked polymers such as crospovidone; effervescent agent such as sodium bicarbonate and citric acid; or mixtures thereof.

Lubricants of the present invention may be selected from talc, magnesium stearate, other alkali earth metal stearate like calcium, zinc etc., lauryl sulphate, hydrogenated vegetable oil, sodium benzoate, sodium stearyl tumarate, glyceryl monostearate and PEG 4000.

Glidants of the present invention may be selected from colloidal silicon dioxide and talc.

Coloring agent of the present invention may be selected from any colorant used in pharmaceuticals which is approved and certified by the FDA. It may include Lake of Tartrazine, Lake of Quinoline Yellow, Lake of Sunset Yellow and Lake of Erythrosine, Lack of Carmosine Ponceau, Allura Red.

The preferred colors for the present invention are Lake of Tartrazine and lake of Quinoline yellow, as these are comparatively cheaper and gives excellent uniformity of color to the dosage form.

The process of the present invention comprises:

Step 1—Treating oxcarbazepine with the wetting agent, which could be achieved by either of the following processes.

(i) by treating powdered oxcarbazepine of desired particle size alone, or a blend of oxcarbazepine and other excipients with a sufficient amount of aqueous solution containing a wetting agent.

(ii) by drying blending wetting agent with oxcarbazepine alone or a mixture of oxcarbazepine and other excipients and granulating with water.

(iii) dry blending wetting agent with oxcarbazepine alone or a mixture of oxcarbazepine and other excipients and granulating by compaction or slugging.

Step 2—Drying the treated mixture, if necessary, by conventional techniques such as spray drying, air drying or flash evaporation.

Step 3—Dried granules/particles are milled, screened or ground, if necessary.

Step 4—Granules/particles of step 3 are compounded with other excipients to formulate the desired dosage form.

The desired dosage form of the present invention could be a tablet, capsule or solution. Most preferred dosage form of the present invention is a tablet which can be produced by using conventional tabletting processes such as dry or wet granulation. The preferred method is wet granulation.

### DETAILED DESCRIPTION OF THE INVENTION

The invention is further illustrated by the following examples but they should not be construed as limiting the scope of this invention in any way.

### EXAMPLES 1 to 4

These examples describe the preparation of oxcarbazepine tablets with four different concentrations of wetting agent (sodium lauryl sulphate).

### EXAMPLES 1-4

Oxcarbazepine tablets with (0.625%-3.75%) wetting agent (sodium lauryl sulphate (SLS))

| | Example 1 | Example 2 | Example 3 | Example 4 |
|---|---|---|---|---|
| Ingredient | (mg/unit) | (mg/unit) | (mg/unit) | (mg/unit) |
| Oxcarbazepine | 600 | 600 | 600 | 600 |
| (Median particle size | | | | |
| 26μ) | | | | |
| Microcrystalline | 122.2 | 117.2 | 107.2 | 97.2 |
| cellulose | | | | |
| Hydroxypropyl methyl | 16.8 | 16.8 | 16.8 | 16.8 |
| cellulose | | | | |
| Sodium lauryl sulphate | 5.0 | 10.0 | 20.0 | 30.0 |
| | (0.625%) | (1.25%) | (2.5%) | (3.75%) |
| Cross-linked | 40.0 | 40.0 | 40.0 | 40.0 |
| polyvinylpyrrolidone | | | | |
| Lake of Tartrazine | 4.0 | 4.0 | 4.0 | 4.0 |
| Colloidal silicon | 3.2 | 3.2 | 3.2 | 3.2 |
| dioxide | | | | |
| Magnesium stearate | 8.8 | 8.8 | 8.8 | 8.8 |
| Total | 800 | 800 | 800 | 800 |

1. Microcrystalline cellulose (about halt the quantity) and hydroxy propyl methyl cellulose are sifted through (60 BSS) sieve and color sifted through (100 bss) sieve; and mixed with oxcarbazepine for about 15 minutes to make a uniform blend.

2. Dry blend of step 1 is granulated with sodium lauryl sulphate solution in water.

3. The wet mass of step 2 is dried in fluidized bed dryer for 15 minutes.

4. The dried material of step 3 is passed through #22 BSS.

5. Cross linked polyvinyl pyrrolidone and microcrystalline cellulose (rest of the quantity) are sieved through #60 BSS and colloidal silicon dioxide is sieved through #44 BSS. These are then mixed with the dried material of step 4.

6. Magnesium stearate is passed through sieve # 60 BSS and mixed with the material of step 5.

7. Lubricated blend of step 6 is compressed using 19×8.8 mm, oval shaped, bioconcave tooling to make the tablets of about 6.6 mm thickness and 800 mg weight.

The tablets prepared by the above composition and process had hardness in the range of 10 to about 15 kp. The disintegration time in water was less than 2 minutes. The oxcarbazepine tablets were tested in three dissolution media i.e. 2% sodium lauryl sulphate in water, 2% sodium lauryl sulphate in 0.1N HCl, and phosphate buffer of pH 6.8 according to the procedure described in the United States Pharmacopoeia XXIII, Apparatus USPII (Paddle) @ 50 rpm and found to have the release given in Tables 1, 2 and 3. For comparison Trileptal®—600 mg (oxcarbazepine tablets) of Novartis are used.

Tables 1 to 3 give comparative dissolution data of batches prepared by the composition and process given in Examples 1 to 4 in three different dissolution media with the marketed oxcarbazepine tablets (Trileptal®) of Novartis. The dissolved oxcarbazopine is expressed in percentage over an elapsed time period in minutes.

TABLE 1
Dissolution profile of oxcarbazepine tablets (prepared by
examples 1-4) in comparison with “Trileptal ®”,
in 2% sodium lauryl sulphate solution in water at 37° C./
50 rpm using apparatus USP II (Paddle)/900 ml.

| Oxcarbazepine | | | | |
|---|---|---|---|---|
| Tablets of | 15 min. | 30 min. | 45 min. | 60 min. |
| Example 1 | 58.2 | 75.8 | 81.9 | 86.4 |
| Example 2 | 62.4 | 80.1 | 86.7 | 92.3 |
| Example 3 | 68.1 | 84.3 | 88.2 | 95.8 |
| Example 4 | 71.2 | 85.8 | 90.1 | 97.1 |
| Novartis | 69.1 | 81.2 | 86.8 | 90.2 |
| (Trileptal ®) | | | | |

Example 1 - oxcarbazepine treated with 0.625% SLS
Example 2 - oxcarbazepine treated with 1.25% SLS
Example 3 - oxcarbazepine treated with 2.50% SLS
Example 4 - oxcarbazepine treated with 3.75% SLS

TABLE 2
Dissolution profile of oxcarbazepine tablets (prepared
by Example 1-4) in comparison with “Trileptal ®”
in 0.1 N HCl containing 2% sodium lauryl sulphate at 37° C.

| Oxcarbazepine | | | | |
|---|---|---|---|---|
| Tablets of | 15 min. | 30 min. | 45 min. | 60 min. |
| Example 1 | 51.1 | 63.2 | 77.3 | 80.0 |
| Example 2 | 53.4 | 65.6 | 80.5 | 82.6 |
| Example 3 | 55.8 | 67.2 | 82.8 | 85.6 |
| Example 4 | 57.3 | 68.9 | 83.5 | 87.1 |
| Novartis | 53.7 | 68.8 | 78.3 | 81.5 |
| (Trileptal ®) | | | | |

Example 1 - oxcarbazepine treated with 0.625% SLS
Example 2 - oxcarbazepine treated with 1.25% SLS
Example 3 - oxcarbazepine treated with 2.50% SLS
Example 4 - oxcarbazepine treated with 3.75% SLS

TABLE 3
Dissolution profile of oxcarbazepine tablets (prepared by
Examples 1-4) in comparison with “Trileptal ®”
in phosphate buffer of pH - 8 at 37° C.

| Oxcarbazepine | | | | |
|---|---|---|---|---|
| Tablets of | 15 min. | 30 min. | 45 min. | 60 min. |
| Example 1 | 67.0 | 76.4 | 80.3 | 83.4 |
| Example 2 | 69.2 | 79.3 | 84.5 | 87.3 |
| Example 3 | 71.3 | 81.6 | 85.3 | 89.2 |
| Example 4 | 73.6 | 83.5 | 86.7 | 90.3 |
| Novartls | 70.0 | 80.6 | 83.8 | 86.5 |
| (Trileptal ®) | | | | |

Example 1 - oxcarbazepine treated with 0.625% SLS
Example 2 - oxcarbazepine treated with 1.25% SLS
Example 3 - oxcarbazepine treated with 2.50% SLS
Example 4 - oxcarbazepine treated with 3.75% SLS

In case of sodium lauryl sulphate it has been observed that satisfactory results are obtained with 0.5 to 5.0% amount of SLS by weight of the composition and preferably from about 2 to 4% by weight.

Table 4 gives dissolution profile of oxcarbazepine tablets prepared with different particle size range of oxcarbazepine without wetting agent in 2% sodium lauryl sulphate in water at 37° C. The dissolved oxcarbazepine expressed in percentage over an elapsed time period in minutes.

TABLE 4
Dissolution profile of oxcarbazepine tablets (prepared
with different particle size of oxcarbazepine) without
wetting agent, in 2% SLS in H₂O at 37° C.

| Tablets | | | | |
|---|---|---|---|---|
| containing | | | | |
| oxcarbazepine | | | | |
| of median | | | | |
| particle size* | 15 min. | 30 min. | 45 min. | 60 min. |
| 1.5μ with 90% | 42.7 | 64.8 | 76.4 | 79.8 |
| <10μ | | | | |
| 9μ with 90% | 70.3 | 82.9 | 87.3 | 91.8 |
| <20μ | | | | |
| 10μ with 90% | 71.6 | 83.7 | 88.2 | 92.2 |
| <30μ | | | | |
| 26μ with 90% | 30.7 | 67.8 | 81.4 | 85.0 |
| <52μ | | | | |
| Novartis | 69.1 | 81.2 | 86.8 | 90.2 |
| Trileptal ® | | | | |

*prepared using the same composition (except SLS) and process as given for Examples 1 to 4.

This data clearly show that oxcarbazepine tablets do not give the desired dissolution profile when prepared without the wetting agent. The median particle size has to be reduced below 10μ for getting the desired dissolution profile.

While the present invention has been described in terms of its specific embodiments, certain modifications and equivalents will be apparent to those skilled in the art and are intended to be included within the scope of the present invention.

We claim:

1. A dosage form composition for oral administration comprising oxcarbazepine and a wetting agent.

2. The composition according to claim 1 wherein the wetting agent is a surface active agent.

3. The composition according to claim 2 wherein the surface active agent is anionic, cationic, or non-ionic.

4. The surface active agent according to claim 3 wherein the anionic surface active agent is selected from the group consisting of sodium laurate, dialkylsodium sulfosuccinates, sodium stearate, potassium stearate, sodium oleate, and mixtures thereof.

5. The surface active agent according to claim 3 wherein the cationic surface active agent is selected from the group consisting of benzalkonium chloride, bis-2-hydroxyethyl oleyl amine, and mixtures thereof.

6. The surface active agent according to claim 3 where in the non-ionic surface active agent is selected from the group consisting of polyoxyethylene sorbitan fatty acid esters, fatty alcohol's, glyceryl esters, and mixtures thereof.

7. The composition according to claim 3 wherein the concentration of surface active agent is from about 0.5% to about 3% by weight of the dosage form.

8. The composition of a dosage form as described in claim 1 further comprising other pharmaceutically acceptable excipients.

9. The composition according to claim 8 where the pharmaceutically acceptable excipients include diluents, binders, disintegrants, lubricants, glidants, coloring agents, flavouring agents, and sweeteners.

10. The composition according to claim 1 wherein the dosage form is a tablet.

11. The composition according to claim 10 wherein the tablet is coated.

12. A process for the preparation of oxcarbazepine dosage form for oral administration comprising the steps of:
(a) treating oxcarbazepine alone or a blend of oxcarbazopine and other pharmaceutical excipients with a wetting agent; and
(b) formulating into a suitable dosage form.

13. The process according to claim 12 wherein step (a) is achieved by wet granulation or dry granulation.

14. The process according to claim 13 wherein the wet granulation is done by aqueous granulation.

15. The process according to claim 13 wherein the dry granulation is done by slugging or compaction.

16. The process according to claim 12 wherein the wetting agent is a surface active agent.

17. The process according to claim 16 wherein surface active agent is anionic, cationic or non-ionic.

18. The process according to claim 17 wherein the anionic surface active agent is selected from the group consisting of sodium lauryl sulphate, sodium laurate, dialkylsodium sulfosuccinates, sodium stearate, potassium stearate, sodium oleate, and mixtures thereof.

19. The process according to claim 17 wherein the cationic surface active agent is selected from the group consisting of benzalkonium chloride, bis-2-hydroxyethyl oleyl amine, and mixtures thereof.

20. The process according to claim 17 wherein the non-ionic surface active agent is selected from the group consisting of polyoxyethylene sorbitan fatty acid esters, fatty alcohols, glyceryl esters, and mixtures thereof.

21. The process according to claim 16 or 17 wherein the wetting agent is sodium lauryl sulphate.

22. The process according to claim 21 wherein the concentration of sodium lauryl sulphate is from about 0.5% to about 5.0% by weight of the total composition.

23. The process according to claim 12 wherein the other pharmaceutical excipients include diluents, binders, disintegrants, lubricants, glidants, coloring agents, flavouring agents and sweeteners.

24. The process according to claim 12 wherein the suitable dosage form is a tablet.

25. The process according to claim 24 wherein the tablet is coated.
